(19) United States
(12) Patent Application Publication
(10) Pub. No.: US 2008/0068454 A1
(43) Pub. Date: Mar. 20, 2008

(54) IMAGE DISPLAY APPARATUS

(75) Inventor: Katsumi HIRAKAWA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/864,486

(22) Filed: Sep. 28, 2007

(30) Foreign Application Priority Data
Apr. 18, 2005 (JP) 2005-119929

(51) Int. Cl. A61B 1/05

(52) U.S. Cl. 348/65; 348/E05.027

(57) ABSTRACT

An image display apparatus according to the present invention includes an image processing controller which acquires an image from an image storage unit, controls various types of image processing on the acquired image, and stores a processed image in the image storage unit, a region detector which detects a feature image region having a predetermined feature from the image, a display mode setting unit which calculates the occupancy rate of the feature image region relative to the entire image region, and associates a predetermined image display mode with the image according to the calculated occupancy rate, and an image display controller which controls image display to sequentially display the series of images stored in the image storage unit based on the image display mode associated with each image.

### BACKGROUND OF THE INVENTION

1. Field of the Invention

The present invention generally relates to an image display apparatus which sequentially displays a series of images supplied as an input, and specifically to an image display apparatus adapted to and suitable for a display of a series of images captured in a body of a subject by a capsule endoscope.

2. Description of the Related Art

In recent years, a swallowable capsule endoscope has been developed in the field of endoscope. The capsule endoscope has an imaging function and a radio communication function. After being swallowed from the mouth of a subject for an observation of various internal organs, the capsule endoscope travels through the interior of a digestive system such as a stomach, a small intestine, and a large intestine following peristaltic movements thereof while sequentially capturing images until naturally discharged therefrom.

While the capsule endoscope travels through the internal organs, image data captured by the capsule endoscope in the body of the subject is sequentially transmitted to the outside of the body by radio communication. The image data is stored in a memory in a receiving device arranged outside the body, or displayed on a monitor provided to the receiving device. Doctor or nurse can make diagnosis according to images presented on a monitor based on the image data stored in the memory, or according to images presented on the monitor of the receiving device simultaneously with the reception of the image data.

Generally, the capsule endoscope captures an enormous amount of images, and the doctor or nurse spends a significant amount of time and energy for observing the images and making diagnosis. In view of the problem, published Japanese translation of PCT international publication for patent application (Kohyo) No. 2004-521662 discloses a display apparatus which determines a degree of similarity between two images and changes a display rate of the images based on the result of determination to facilitate the observation of the displayed images presented based on the image data stored in the memory. The display apparatus disclosed focuses on the fact that a large amount of similar images are sequentially captured when the movement of the capsule endoscope becomes sluggish, and displays the images at a low display rate when the degree of similarity between two images is low, whereas displays the images at a high display rate when the degree of similarity is high.

### SUMMARY OF THE INVENTION

An object of the present invention is to at least solve the problems as described above.

An image display apparatus according to one aspect of the present invention includes a region detecting unit that detects a feature image region having a predetermined feature from each image included in a series of images, an associating unit that calculates an occupancy rate of the feature image region relative to an entire image region of the each image to associate a predetermined image display mode with the each image according to the occupancy rate calculated, and a display control unit that controls to sequentially display the series of images based on the image display mode associated with the each image.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram of a configuration of an image display apparatus according to a first embodiment of the present invention;

FIG. 2 is a flowchart of a process procedure of the image display apparatus shown in FIG. 1;

FIG. 3 is a flowchart of a process procedure of an image display process shown in FIG. 2;

FIG. 4 is a view of an example of a GUI screen displayed by the image display apparatus shown in FIG. 1;

FIG. 5 is a block diagram of a configuration of an image display apparatus according to a second embodiment of the present invention;

FIG. 6 is a flowchart of a process procedure of the image display apparatus shown in FIG. 5;

FIG. 7 is a flowchart of a process procedure of an image display process shown in FIG. 6;

FIG. 8 is a block diagram of a configuration of an image display apparatus according to a third embodiment of the present invention;

FIG. 9 is a flowchart of a process procedure of the image display apparatus shown in FIG. 8; and

FIG. 10 is a flowchart of a process procedure of an image display process shown in FIG. 9.

### DETAILED DESCRIPTION OF THE PREFERRED EMBODIMENTS

Exemplary embodiments of an image display apparatus according to the present invention will be described in detail below with reference to the accompanying drawings. It should be noted that the present invention is not limited to the embodiments. The same reference character is used to denote the same element in the drawings.

### First Embodiment

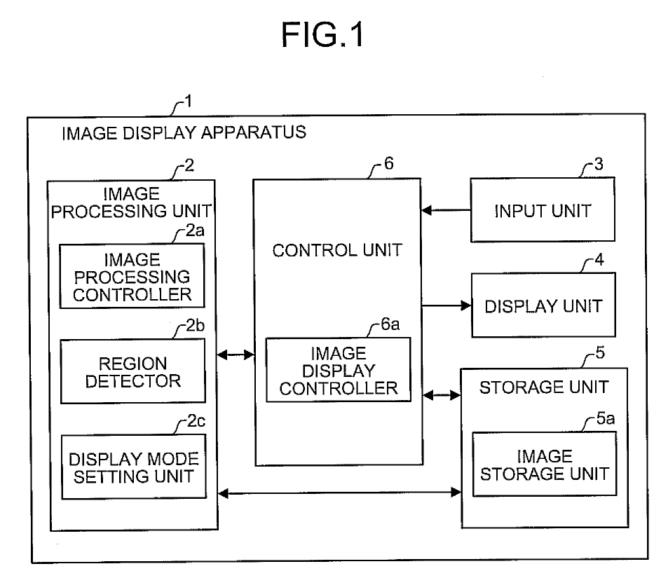

An image display apparatus according to the first embodiment of the present invention will be described. FIG. 1 is a block diagram of a configuration of an image display apparatus 1 according to the first embodiment. As shown in FIG. 1, the image display apparatus 1 includes an image processing unit 2 that processes an image stored in a storage unit 5, an input unit 3 that receives inputs of various types of information, a display unit 4 that displays various types of information, the storage unit 5 that stores various types of information, and a control unit 6 that controls a process and an operation of each unit in the image display apparatus 1. The image processing unit 2, the input unit 3, the display unit 4, and the storage unit 5 are electrically connected to the control unit 6.

The image processing unit 2 includes an image processing controller 2a, a region detector 2b, and a display mode setting unit 2c which serves as an associating unit. The image processing controller 2a acquires an image from the storage unit 5, controls various types of image processing on the acquired image, and outputs the processed image to the storage unit 5 for storage. The image processing controller 2a controls the region detector 2b and the display mode setting unit 2c in particular, to execute predetermined image processing.

The region detector 2b detects a feature image region in the image acquired by the image processing controller 2a. The feature image region is an image region which has a predetermined feature. The region detector 2b detects the feature image region by distinguishing the predetermined feature based on color information of each pixel constituting the image. When there are plural features to be detected, the region detector 2b detects the feature image region corresponding to each feature. The region detector 2b may detect the feature image region based on various feature quantities, such as a contour, texture, and concentration gradient, other than color information. Alternatively, the region detector 2b may divide the image as a whole into sub regions having a specific feature in advance, before extracting a region corresponding to the predetermined feature.

The display mode setting unit 2c calculates the occupancy rate of the feature image region detected by the region detector 2b relative to the entire image region, and associates the processed image with a predetermined image display mode according to the calculated occupancy rate. The display mode setting unit 2c refers to a previously-input threshold of the occupancy rate in particular, and attaches displayability information which indicates whether the image is to be displayed or not to the processed image according to the magnitude relation of the threshold and the calculated occupancy rate. Specifically, the displayability information includes display information which indicates that the image is to be displayed, and skip information which indicates that the image is not to be displayed. The display mode setting unit 2c attaches one of the display information and the skip information to the processed image.

Further, when there are plural feature image regions to be detected from a single image, the display mode setting unit 2c calculates the occupancy rate of each feature image region. Based on the sum and the difference of the calculated plural occupancy rates, the maximum value and the minimum value of the calculated plural occupancy rates, and other various statistical values, the display mode setting unit 2c attaches displayability information to the processed image according to at least one of the occupancy rates. A condition of determination of the magnitude relation between the occupancy rate and the threshold, a condition of selection of a specific occupancy rate to be referred to from plural occupancy rates, and the like are set in advance according to the characteristic of the processed image.

The input unit 3 receives an input of an image to be processed by the image display apparatus 1, various types of processing information, and the like. Specifically, the input unit 3 is provided with a communication interface such as USB and IEEE1394, and receives the input of images from an external device. Further, the input unit 3 is provided with various switches, an input key, a mouse, a touch panel, and the like, so as to receive the input of various types of processing information, such as information related to the feature of the image region to be detected by the region detector 2b, the threshold to be referred to by the display mode setting unit 2c, and the condition of determination of the magnitude relation between the threshold and the occupancy rate. The input unit 3 may be provided with an interface compatible with a portable recording medium such as a CompactFlash®, so as to receive the input of an image from the portable recording medium.

The display unit 4 is provided with, for example, a liquid crystal display so as to display various types of information including the image. The display unit 4 displays, in particular, an image stored in the storage unit 5 and a Graphical User Interface (GUI) screen which prompts the user of the image display apparatus 1 to input various types of processing information.

The storage unit 5 is implemented with a read only memory (ROM) which stores various types of processing programs and the like in advance, and a random access memory (RAM) which stores process parameters of each process, processed data, and the like. The storage unit 5 is specifically provided with an image storage unit 5a which serves as a storage area where an externally-input image and an image processed by the image processing unit 2 are stored. The storage unit 5 may be an image storage unit to which a portable recording medium such as a CompactFlash® can be detachably attached.

The control unit 6 is implemented with a central processing unit (CPU) or the like that executes various types of processing programs stored in the storage unit 5. The control unit 6 is provided with an image display controller 6a, which makes a series of images stored in the image storage unit 5a with the displayability information attached sequentially displayed on the display unit 4 based on the displayability information of each image.

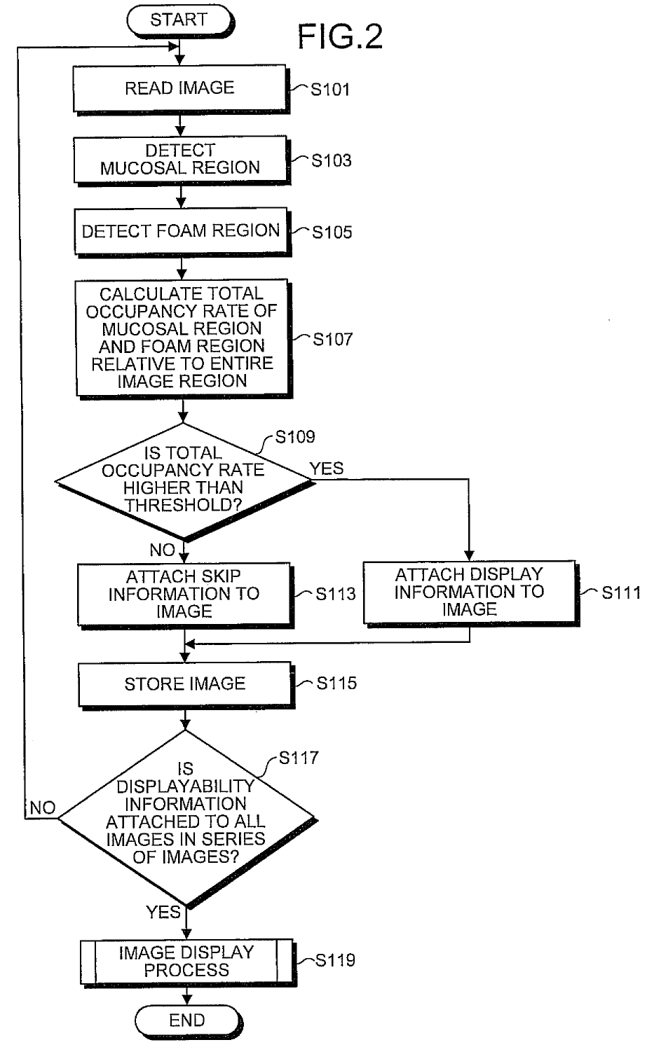

Processes and operations performed by the image display apparatus 1 will be explained. FIG. 2 is a flowchart of a process procedure performed by the image display apparatus 1 to process and display a series of images stored in the image storage unit 5a under the control of the control unit 6. The flowchart of FIG. 2 shows a process procedure of displaying a series of images which are captured and generated by a capsule endoscope (not shown) and which cover a mucosa and foreign substances in a small intestine.

As shown in FIG. 2, the image processing controller 2a reads a temporally first image from the series of images stored in the image storage unit 5a (step S101). The region detector 2b detects a mucosal region which indicates the mucosa of an inner lining of the small intestine (step S103), and a foam region which indicates a foam as a foreign substance present in the small intestine (step S105) from the read images as feature image regions.

Then, the display mode setting unit 2c calculates the occupancy rate relative to the entire image region of each of the mucosal region and the foam region detected by the region detector 2b, and calculates the sum of the occupancy rates as a total occupancy rate (step 5107) to determine whether the calculated total occupancy rate is higher than a threshold which is input in advance (step S109). When the total occupancy rate is higher than the threshold (Yes in step S109), the display mode setting unit 2c attaches the display information to the processed image (step S111), whereas when the total occupancy rate is equal to or lower than the threshold (No in step S109), the display mode setting unit 2c attaches the skip information to the processed image (step S113).

Then, the image processing controller 2a stores the image to which the displayability information is attached in the image storage unit 5a (step S115), and checks whether the displayability information is attached to all the images included in the series of images (step S117). When the displayability information has not been attached to all the images (No in step S117), the image processing controller 2a repeats the process from step S101 for an image to which the displayability information has not been attached.

When the displayability information is attached to all the images (Yes in step S117), the image display controller 6a executes an image display process according to which the series of images to which the displayability information is attached and which are stored in the image storage unit 5a are displayed sequentially based on the displayability information of each image (step S119) to end the series of processing.

Since the skip information is attached to an image which has a low total occupancy rate of the mucosal region and the foam region, the display of such an image in which the occupancy rate of the mucosal region is low and therefore the necessity of observation thereof is low can be skipped. Thus, time required for the observation of the series of images can be shortened, and only those images for which the observation is necessary can be effectively observed. The necessity of observation is determined based on the total occupancy rate of the mucosal region and the foam region because the observation of mucosa may be possible when the foams are present on the mucosa due to a slight transparency of the foams.

In step S103, the region detector 2b detects an image region in a color range from red to skin color as the mucosal region, and in step S105, detects an image region of white color as the foam region. In step S107, the display mode setting unit 2c may calculate the total occupancy rate by calculating the total amount of the mucosal region and the foam region, and then calculating the occupancy rate relative to the entire image region, instead of calculating the sum of occupancy rates of the mucosal region and the foam region.

In step S115, the image processing controller 2a may store the image to which the displayability information is attached so as to update the previously stored image in the image storage unit 5a. Alternatively, the image processing controller 2a may store the image to which the displayability information is attached anew leaving the previously stored image as it is. Alternatively, the image processing controller 2a may store association information which associates the displayability information with a corresponding image in the storage unit 5.

In the processing shown in the flowchart of FIG. 2, the image to which the displayability information has not been attached is read and the displayability information is attached thereto. Alternatively, the image to which the displayability information has already been attached may be read and the displayability information may be updated in a similar process procedure. In the processing shown in the flowchart of FIG. 2, which of the display information and the skip information is to be set is determined based on the total occupancy rate of the mucosal region and the foam region. Additionally, the occupancy rate of each of the mucosal region and the foam region may be considered in the determination.

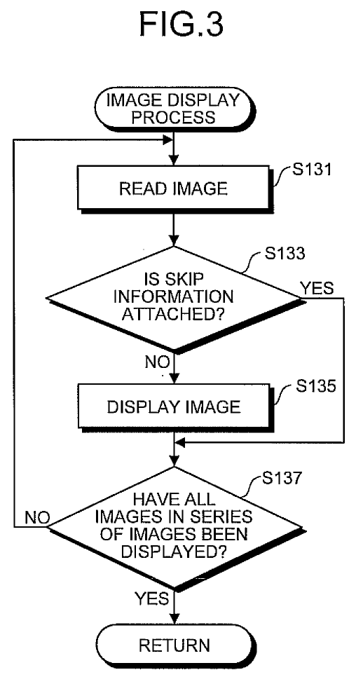

The image display process of step S119 shown in FIG. 2 will be explained. FIG. 3 is a flowchart of the process procedure of the image display process. As shown in FIG. 3, the image display controller 6a reads the temporally first image from the series of images to which the displayability information is attached and which are stored in the image storage unit 5a (step S131), and determines whether the attached displayability information is the skip information or not (step S133).

When the skip information is not attached (No in step S133), the image display controller 6a displays the read image on the display unit 4 (step S135), and determines whether all the images included in the series of images have been displayed or not (step S137). On the other hand, when the skip information is attached (Yes in step S133), the image display controller 6a proceeds to the process of step S137 without displaying the read image.

If all the images of the series of images have not been displayed (No in step S137), the image display controller 6a repeats the process from step S131 on a yet-displayed image. When all the images of the series of images are displayed (Yes in step S137), the process returns to step S119. Thus, the image display controller 6a sequentially displays the images of the series of images according to the displayability information attached to each image.

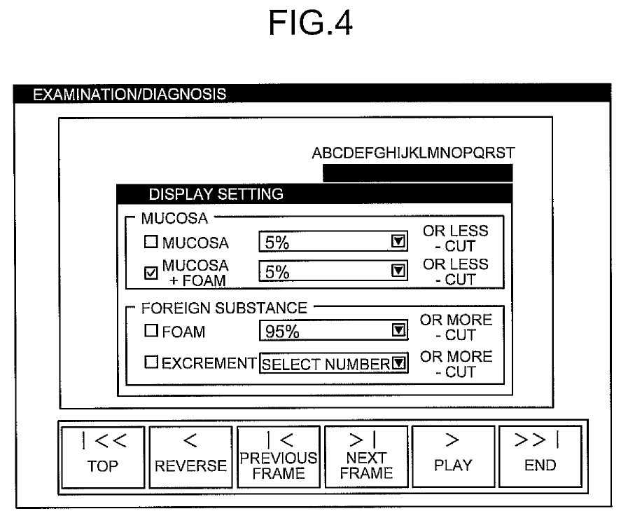

FIG. 4 is a view of an example of a GUI screen which is displayed at a time when the threshold of the occupancy rate of the feature image region is input. In the GUI screen shown in FIG. 4, processing conditions concerning the mucosa and the foreign substances corresponding to the feature image regions are shown in a “display setting” window. Various processing conditions can be set by checking a check box, inputting from a key, or selecting an item from a pull-down menu. In the example shown in FIG. 4, a condition is set in such a manner that “MUCOSA+FOAM: 5% OR LESS−CUT”. Based on the set condition, the display mode setting unit 2c attaches the skip information to the processed image when the total occupancy rate of the mucosal region and the foam region is 5% or less.

As explained above, in the image display apparatus 1 according to the first embodiment: the region detector 2b detects the feature image region having a predetermined feature related to the mucosa, the foreign substance, and the like from each image included in the series of images stored in the image storage unit 5a; the display mode setting unit 2c calculates the occupancy rate of the feature image region relative to the entire image region, and attaches the displayability information such as the display information and the skip information according to the calculated occupancy rate; and the image display controller 6a displays the series of images based on the displayability information attached to each image. Therefore, the display of an image in which the occupancy rate of the mucosal region is low and for which the necessity of observation is low can be skipped, whereby the time required for the observation of the series of images can be shortened, and images for which the observation is necessary can be efficiently observed.

### Second Embodiment

A second embodiment of the present invention will be explained. In the first embodiment explained above, the displayability information is attached to each image according to the occupancy rate of the feature image region. In the second embodiment, a display rate is set for each image.

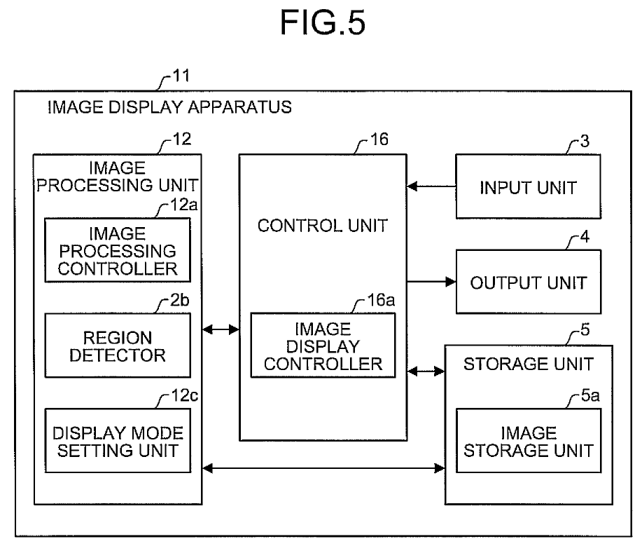

FIG. 5 is a block diagram of a configuration of an image display apparatus 11 according to the second embodiment. As shown in FIG. 5, the image display apparatus 11 includes an image processing unit 12 and a control unit 16 in place of the image processing unit 2 and the control unit 6 of the image display apparatus 1. Further, the image processing unit 12 includes an image processing controller 12a and a display mode setting unit 12c in place of the image processing controller 2a and the display mode setting unit 2c of the image processing unit 2, and the control unit 16 includes an image display controller 16a in place of the image display controller 6a of the control unit 6. In other respects, the configuration of the image display apparatus according to the second embodiment is the same as that of the first embodiment, and the same element is denoted by the same reference character.

The image processing controller 12a, similarly to the image processing controller 2a, acquires an image stored in the image storage unit 5a, processes the acquired image, and stores the processed image in the image storage unit 5a. The image processing controller 12a, however, controls the display mode setting unit 12c instead of the display mode setting unit 2c to associate each image acquired from the image storage unit 5a with a display mode.

The display mode setting unit 12c, similarly to the display mode setting unit 2c, calculates the occupancy rate of the feature image region to associate the processed image with a predetermined image display mode according to the calculated occupancy rate. The display mode setting unit 12c sets a display rate which indicates the speed of image display as the image display mode instead of setting the displayability information. Specifically, the display rate includes a normal display rate which indicates a normal speed of image display, a high display rate which indicates a faster display rate than the normal display rate, and a low display rate which indicates a slower display rate than the normal display rate. The display mode setting unit 12c sets one of the normal display rate, the high display rate, and the low display rate to the processed image.

The image display controller 16a displays the series of images to which the display rate is set and which are stored in the image storage unit 5a sequentially on the display unit 4 according to the display rate of each image.

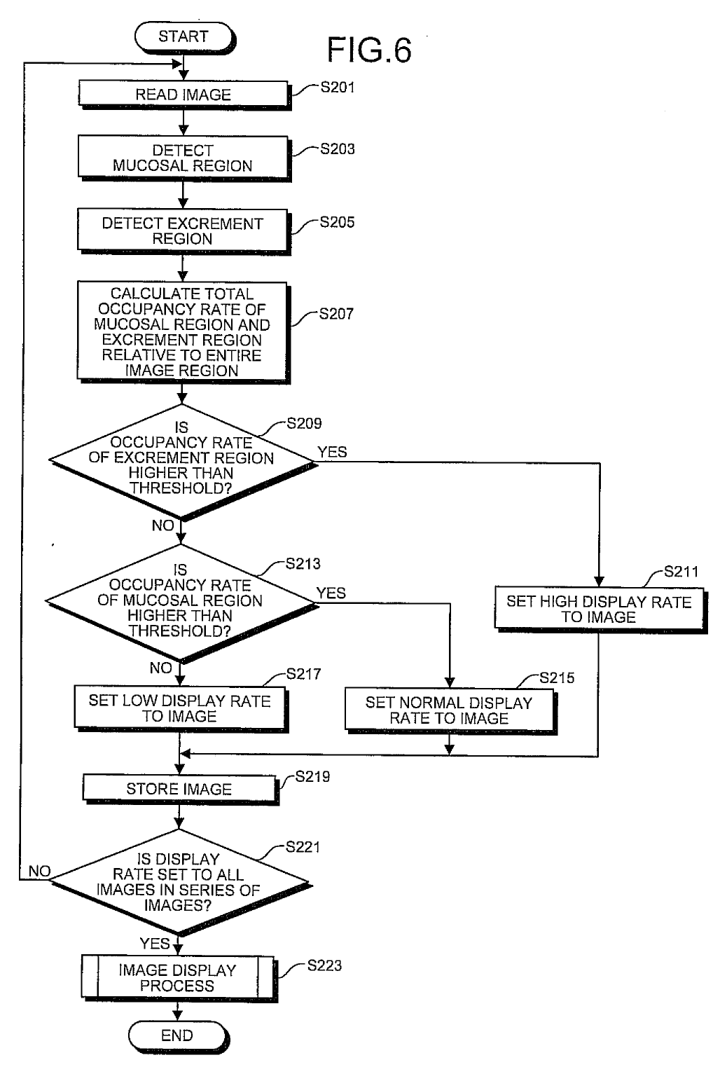

Processes and operations performed by the image display apparatus 11 will be explained. FIG. 6 is a flowchart of a process procedure performed by the image display apparatus 11 to process and display the series of images stored in the image storage unit 5a under the control of the control unit 16. The flowchart of FIG. 6 shows a process procedure of displaying a series of images which are captured and generated by a capsule endoscope (not shown) and which cover a mucosa and foreign substances in a small intestine.

As shown in FIG. 6, the image processing controller 12a reads a temporally first image from the series of images stored in the image storage unit 5a (step S201). The region detector 2b detects a mucosal region which indicates the mucosa of an inner lining of the small intestine (step S203), and an excrement region which indicates an excrement as a foreign substance present in the small intestine (step S205) from the read image as feature image regions. In step S205, the region detector 2b detects an image region of brown color as the excrement region.

Then, the display mode setting unit 12c calculates the occupancy rate relative to the entire image region of each of the mucosal region and the excrement region detected by the region detector 2b (step S207) to determine whether the calculated occupancy rate of the excrement region is higher than a threshold which is input in advance for the excrement region (step S209). When the occupancy rate of the excrement region is higher than the threshold (Yes in step S209), the display mode setting unit 12c sets the high display rate to the processed image (step S211).

When the occupancy rate of the excrement region is equal to or lower than the threshold (No in step S209), the display mode setting unit 12c further determines whether the occupancy rate of the mucosal region is higher than a threshold input in advance for the mucosal region (step S213). When the occupancy rate of the mucosal region is higher than the threshold (Yes in step S213), the display mode setting unit 12c sets the normal display rate to the processed image (step S215), whereas when the occupancy rate of the mucosal region is equal to or lower than the threshold (No in step S213), the display mode setting unit 12c sets the low display rate to the processed image (step S217).

The image processing controller 12a stores the image to which the display rate is set in the image storage unit 5a (step S219), and determines whether the display rate is set to all the images included in the series of images (step S221). If the display rate has not been set to all the images (No in step S221), the image processing controller 12a repeats the process from step S201 for an image to which the display rate has not been set.

When the display rate is set to all the images (Yes in step S221), the image display controller 16a executes the image display process in which the series of images to which the display rate is set and which are stored in the image storage unit 5a are sequentially displayed based on the display rate of each image (step S233), to end the series of processes.

When the high display rate is set to an image in which the occupancy rate of the excrement region is high, a display time of an image for which the necessity of observation is low can be reduced and the observation time can be shortened. Further, when the low display rate is set to an image in which the occupancy rate of the mucosal region is low, a display time of an image for which the necessity of observation is high and the observation is difficult can be lengthened to ensure the observation. Thus, the series of images can be observed efficiently. The low display rate is set to the image in which the occupancy rate of the mucosal region is low, because it is expected that the foreign substances other than excrement covers the mucosa to make the observation difficult.

In the process shown in the flowchart of FIG. 6, an image for which the display rate has not been set is read and the display rate is set therefor. Alternatively, in a similar process procedure, an image for which the display rate has already been set may be read and the display rate thereof may be updated. Further, in the process shown in the flowchart of FIG. 6, which of the normal display rate, the high display rate, and the low display rate is to be set is determined according to the occupancy rate of each of the mucosal region and the excrement region. Alternatively, however, occupancy rate of a region which shows a foreign substance such as residue other than excrement may additionally be considered in the determination.

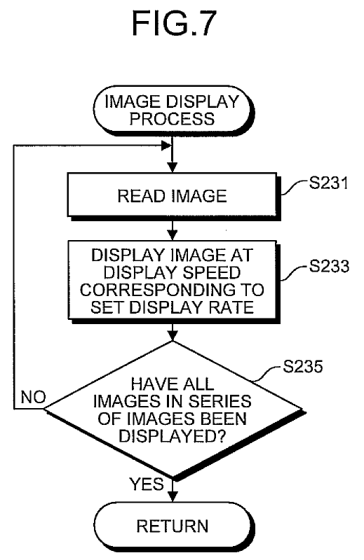

The image display process of step S223 shown in FIG. 6 will be explained. FIG. 7 is a flowchart of the process procedure of the image display process. As shown in FIG. 7, the image display controller 16a reads the temporally first image of the series of images for which the display rate is set and which are stored in the image storage unit 5a (step S231), and displays the read image at a display speed corresponding to the set display rate (step S233).

Thereafter, the image display controller 16a determines whether all the images included in the series of images are displayed or not (step S235). If all the images included in the series of images have not been displayed (No in step S235), the image display controller 16a repeats the process from step S231 on a yet-displayed image, whereas if all the images have been displayed (Yes in step S235), the image display controller 16a returns to step S223. Thus, the image display controller 16a sequentially displays the images of the series of images according to the display rate set for each image.

As explained above, in the image display apparatus 11 according to the second embodiment: the region detector 2b detects the feature image region having a predetermined feature such as mucosa and foreign substances from each image included in the series of images stored in the image storage unit 5a; the display mode setting unit 12c calculates the occupancy rate of the feature image region relative to the entire image region, and sets one of the normal display rate, the high display rate, and the low display rate according to the calculated occupancy rate; and the image display controller 16a displays the series of images based on the display rate set for each image. For example, when the high display rate is set to an image having a high occupancy rate of the excrement region, the display time of an image for which the necessity of observation is low can be reduced, and the observation time can be shortened. Further, when the low display rate is set to an image having a low occupancy rate of the mucosal region, the display time of an image for which the necessity of the observation is high and the observation is difficult can be lengthened to secure observation. Thus, the series of images can efficiently be observed.

### Third Embodiment

A third embodiment of the present invention will be explained. In the second embodiment explained above, the display rate is set for each image in three levels (i.e., low display rate, normal display rate, or high display rate) according to the occupancy rate of the feature image region. In the third embodiment, the display rate is set to each image in such a manner that the display rate successively increases/decreases according to the occupancy rate of the feature image region.

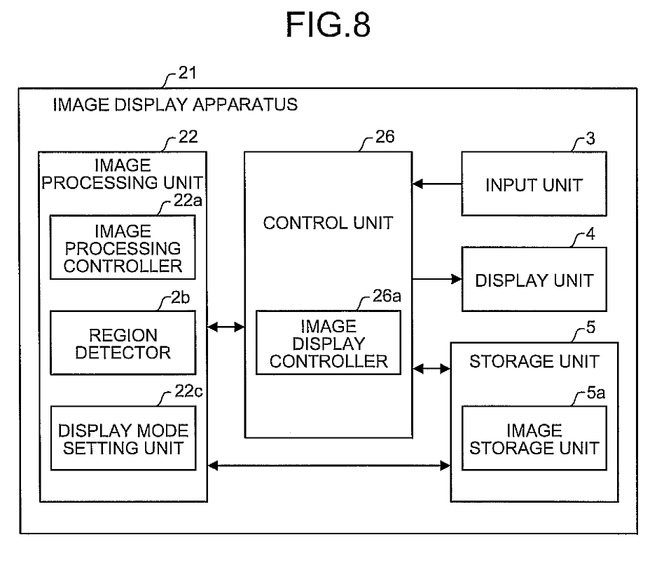

FIG. 8 is a schematic block diagram of a configuration of an image display apparatus according to the third embodiment of the present invention. As shown in FIG. 8, an image display apparatus 21 according to the third embodiment includes an image processing unit 22 in place of the image processing unit 12 of the image display apparatus 11 according to the second embodiment explained above, and a control unit 26 in place of the control unit 16. The image processing unit 22 includes an image processing controller 22a in place of the image processing controller 12a of the image processing unit 12 of the second embodiment explained above, and a display mode setting unit 22c in place of the display mode setting unit 12c. Further, the control unit 26 includes an image display controller 26a in place of the image display controller 16a of the control unit 16 of the second embodiment explained above. In other respects, the configuration of the image display apparatus according to the third embodiment is the same as that of the second embodiment, and the same element is denoted by the same reference character.

The image processing controller 22a controls the display mode setting unit 22c so as to associate each image acquired from the image storage unit 5a with the display mode, and stores each image with which the display mode is made associated by the display mode setting unit 22c in the image storage unit 5a. Other functions of the image processing controller 22a are the same as those of the image processing controller 12a of the image display apparatus 11 according to the second embodiment explained above.

The display mode setting unit 22c, similarly to the display mode setting unit 12c of the image display apparatus 11 according to the second embodiment explained above, calculates the occupancy rate of the feature image region in the image and associates a predetermined image display mode with the processed image according to the calculated occupancy rate. Specifically, the display mode setting unit 22c sets the display rate which successively increases/decreases according to the occupancy rate of the feature image region detected by the region detector 2b relative to the image to each of the processed images. The display mode setting unit 22c calculates a display rate coefficient T1 which successively increases/decreases according to the occupancy rate of the feature image region, and associates the calculated display rate coefficient T1 and a normal display rate set in advance with each of the processed images.

The display rate coefficient T1 is a coefficient which adjusts the display rate in such a manner that the display time of the image increases as the occupancy rate of the feature image region increases, and that the display time of the image decreases as the occupancy rate of the feature image region decreases. The display rate coefficient T1 successively increases/decreases linearly or non-linearly according to the occupancy rate of the feature image region relative to the entire image. The display rate of the processed image is calculated by dividing the normal display rate set in advance by the display rate coefficient T1. The normal display rate is a normal speed of image display. The display mode setting unit 22c sets the normal display rate based on information supplied from the input unit 3 in advance. The normal display rate is managed by the image processing controller 22a as a reference display rate used for setting the display rate of each image.

The image display controller 26a sequentially displays the images on the display unit 4 based on the display rate set for each image by the display mode setting unit 22c as mentioned above. Here, the image display controller 26a calculates the display rate based on the display rate coefficient T1 and the normal display rate associated with an image read from the image storage unit 5a for display, and displays the image on the display unit 4 at the calculated display rate. Other functions of the image display controller 26a are the same as those of the image display controller 16a of the image display apparatus 11 according to the second embodiment explained above.

The control unit 26 provided with the image display controller 26a controls the processes and the operations of the image processing unit 22, and controls the input unit 3, the display unit 4, and the storage unit 5, similarly to the control unit 6 of the image display apparatus 1 of the first embodiment.

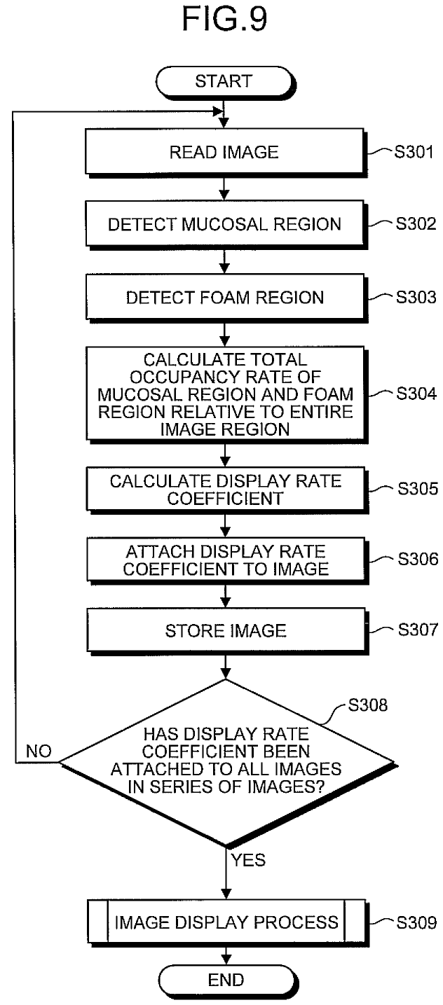

Processes and operations performed by the image display apparatus 21 will be explained. FIG. 9 is a flowchart of a process procedure performed by the image display apparatus 21 to process and display the series of images stored in the image storage unit 5a under the control of the control unit 26. The flowchart of FIG. 9 shows a process procedure of displaying a series of images which are captured and generated by a capsule endoscope (not shown) and which cover a mucosa and foreign substances in a small intestine.

As shown in FIG. 9, the image processing controller 22a reads a temporally first image from the series of images stored in the image storage unit 5a (step S301). The region detector 2b detects a mucosal region which indicates the mucosa of an inner lining of the small intestine (step S302), and a foam region which indicates a foam as a foreign substance present in the small intestine (step S303) from the read image as feature image regions.

The display mode setting unit 22c calculates the occupancy rate of each of the mucosal region and the foam region detected by the region detector 2b relative to the entire image region, and calculates the total occupancy rate as the sum of the calculated occupancy rates (step S304). Then, the display mode setting unit 22c calculates the display rate coefficient T1 which successively increases/decreases according to the total occupancy rate calculated in step S304 (step S305). In step S305, the display mode setting unit 22c calculates the display rate coefficient T1 for each image based on an arithmetic expression set in advance as shown by a formula (1). In the following formula (1), the total occupancy rate x of the feature image region takes a continuous value within a range of 0% to 100%.

Display Rate Coefficient T1=2.0/(1÷exp(−(x−50))) (1)

Thereafter, the display mode setting unit 22c attaches the display rate coefficient T1 set as explained above and the normal display rate set in advance to the processed image (step S306) to associate the display rate coefficient T1 and the normal display rate with the processed image. Thus, the display mode setting unit 22c finishes a display-rate setting process for the processed image.

The display rate of the processed image to which the display rate coefficient T1 is attached is a quotient which is obtained by dividing the normal display rate by the attached display rate coefficient T1. The display rate decreases (i.e., becomes slower) successively along with the increase in the total occupancy rate x, and increases (i.e., becomes faster) successively along with the decrease in the total occupancy rate x. For example, when the total occupancy rate x is 100%, the display rate becomes slower to ½ the normal display rate based on the formula (1). When the total occupancy rate x is 50%, the display rate is equal to the normal display rate based on the formula (1). Further, when the total occupancy rate x is 0%, in other words, when there is little necessity to observe the pertinent image, the display rate is maximized within the controllable range of the image display controller 26a.

The image processing controller 22a stores the image to which the display rate is set in step S306 in the image storage unit 5a (step S307), and subsequently determines whether the display rate coefficient T1 is attached to all the images included in the series of images (step S308). When the display rate coefficient T1 is not attached to all the images (No in step S308), the image processing controller 22a repeats the process from step S301 on the remaining images to which the display rate coefficient T1 has not been set.

On the other hand, when the display rate coefficient T1 is attached to all the images (Yes in step 308), the image display controller 26a executes the image display process in which the series of images to which the display rate coefficient T1 is attached and which are stored in the image storage unit 5a are sequentially displayed at the display rate of each image calculated by dividing the normal display rate by the display rate coefficient T1 of each image (step S309), and finishes the series of processing.

Thus, when the display rate which is calculated by dividing the normal display rate by the display rate coefficient T1 which successively increases/decreases according to the total occupancy rate x of the mucosal region and the foam region is set to each image, and the display rate of the image is successively increased along with the decrease in the total occupancy rate x, the display rate of the image can be successively increased as the occupancy rate of the mucosal region decreases, in other words, as the necessity of observation decreases. Thus, the display time of an image for which the necessity of observation is low can be reduced, and the time required for the observation of the series of images can be shortened. Further, when the display rate of the image is successively increased along with the decrease in the necessity of observation, the display time of the image can be gradually decreased as the necessity of observation decreases. As a result, the change of display rate from the normal display rate to a faster rate is prevented from giving a sense of discomfort to the observer (in other words, a sudden increase in the display rate is prevented so as not to make it difficult for the observer to visually recognize the image).

Further, since the display rate of the image is successively decreased along with the increase in the total occupancy rate x, the display rate of the image can be successively slowed as the occupancy rate of the mucosal region increases, in other words, as the necessity of observation increases. Therefore, the display time of an image for which the necessity of observation is low can be gradually reduced, while the display time of an image for which the necessity of observation is high can be gradually lengthened. As a result, the change of display rate from the normal display rate to a slower rate can be prevented from giving a sense of discomfort to the observer (in other words, a sudden decrease in the display rate is prevented from making it difficult for the observer to visually recognize the image). At the same time, only those images for which the observation is necessary can efficiently and securely be observed.

In step S302, the region detector 2b detects an image region of a color range from red to skin color as the mucosal region. In step S303, the region detector 2b detects an image region of white color as the foam region.

In step S304, the display mode setting unit 22c may calculate the occupancy rate of the sum of the mucosal region and the foam region relative to the entire image region as the total occupancy rate, instead of adding the occupancy rate of the mucosal region and the occupancy rate of the foam region.

Further, in step S307, the image processing controller 22a may store the image to which the display rate coefficient T1 is attached so as to update the previously-stored image in the image storage unit 5a. Alternatively, the image processing controller 22a may store the image to which the display rate coefficient T1 is attached anew leaving the previously-stored image as it is. Further, association information which associates the display rate coefficient T1 with the image may be stored in the storage unit 5.

In the process of the flowchart shown in FIG. 9, an image to which the display rate coefficient T1 has not been attached is read and the display rate coefficient T1 is attached. Alternatively, in a similar process procedure, an image to which the display rate coefficient T1 has already been attached may be read and the display rate coefficient T1 may be updated. Further, in the process of the flowchart shown in FIG. 9, the display rate coefficient T1 is increased/decreased according to the total occupancy rate x of the mucosal region and the foam region. Alternatively, however, the display rate coefficient T1 may be increased/decreased according to at least one of the occupancy rates of the mucosal region and the foam region.

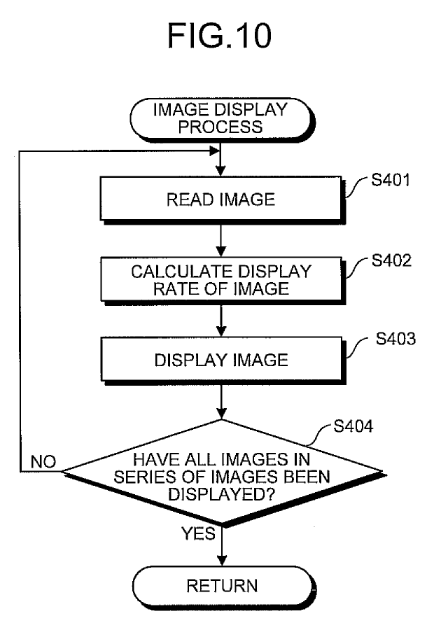

The image display process of step S309 shown in FIG. 9 will be explained. FIG. 10 is a flowchart of a process procedure of the image display process of the image display apparatus 21 according to the third embodiment. As shown in FIG. 10, the image display controller 26a reads a temporally first image of the series of images to which the display rate coefficient T1 is attached and which are stored in the image storage unit 5a (step S401), and calculates the display rate of the read image (step S402).

In step S402, the image display controller 26a extracts the normal display rate and the display rate coefficient T1 attached to the read image, and divides the extracted normal display rate by the extracted display rate coefficient T1 to calculate the display rate of the pertinent image.

Then, the image display controller 26a displays the image read in step S401 on the display unit 4 at the display speed corresponding to the calculated display rate (step S403). For example, when the total occupancy rate x of the feature image region within the pertinent image is 100%, the image display controller 26a displays the image over twice as long time as the display time at the normal display rate on the display unit 4. When the total occupancy rate x is 50%, the image display controller 26a displays the image over the same display time as at the normal display rate on the display unit 4. Further, when the total occupancy rate x is 0%, the image display controller 26a displays the image on the display unit 4 at the highest controllable display rate (i.e., at the display rate at which the display time is substantially zero second).

Thereafter, the image display controller 26a determines whether all the images included in the series of images are displayed or not (step S404). When at least one of the images in the series of images is not displayed (No in step S404), the image display controller 26a repeats the process from step S401 on a remaining yet-displayed image. When all the images in the series of images have been displayed (Yes in step S404), the image display controller 26a returns to the step S309. Thus, the image display controller 26a sequentially displays the series of images at the display rate which increases/decreases according to the occupancy rate of the feature image region relative to the entire image.

As explained above, in the image display apparatus 21 according to the third embodiment: the region detector 2b detects the feature image region having a predetermined feature such as mucosa and foreign substances from each image in the series of images stored in the image storage unit 5a; the display mode setting unit 22c calculates the occupancy rate of the feature image region relative to the entire image region and the display rate coefficient T1 which successively increases/decreases according to the occupancy rate, attaches the calculated display rate coefficient T1 to each image, and sets the display rate which successively increases/decreases according to the increase/decrease of the display rate coefficient T1, in other words, according to the increase/decrease of the occupancy rate of the feature image region for each image; and the image display controller 26a calculates the display rate of each image by dividing the normal display rate by the display rate coefficient T1 attached to each image, and displays the series of images at the display rate calculated for each image. Therefore, the display rate of the image can be successively increased as the occupancy rate of the feature image region decreases, in other words, as the necessity of observation decreases. At the same time the display rate of the image can be successively decreased as the occupancy rate of the feature image region increases, in other words, as the necessity of observation increases. Thus, the display time of the image for which the necessity of observation is low can be gradually shortened so that the time required for the observation of the series of images can be shortened. At the same time, the display time of the image for which the necessity of observation is high can be gradually lengthened. As a result, the change of display rate to a faster rate or to a slower rate is prevented from giving a sense of discomfort to the observer, in other words, the sudden increase/decrease of display rate would not make it difficult for the observer to visually recognize the image. Further, only those images for which the observation is necessary can efficiently and securely be observed.

Further, since the display rate of the image is successively changed as the occupancy rate of the feature image region increases/decreases, the images of high degree of similarly can be displayed in a short time at a high display rate without the need of visual recognition of the degree of similarly between sequential images in the series of images, whereby the display time of the images of high similarity for which the repetitious observation is not necessary can be shortened with ease.

In the first and second embodiments, the image display controller 6a, 16a sequentially displays the series of images from the temporally first image, and displays all the images included in the series of images. Alternatively, however, the image display controller 6a, 16a may start the display from an intermediate image in time series of the series of images based on instruction information input in advance concerning a first-displayed image, or may finish the image display process when completing the display up to an intermediate image in time series of the series of images based on instruction information input in advance concerning a last-displayed image.

Further, in the third embodiment, the display rate which successively increases/decreases according to the occupancy rate of the feature image region relative to the entire image is set for each image. Alternatively, the display time of images included in a group of time-serially successive images of a predetermined number may be averaged, and the image which is time-serially in the middle of the group of the images may be displayed over the average display time. In this case, the image display controller 26a averages the display time of respective images included in the group of time-serially successive images of the predetermined number including an image to be displayed (for example, a group of successive images including eleven images in total, i.e., the image to be displayed and previous five images and subsequent five images) based on the display rate coefficient T1 and the normal display rate, and displays the image to be displayed on the display unit 4 over the averaged display time.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image display apparatus comprising:
a region detecting unit that detects a feature image region having a predetermined feature from each image included in a series of images;
an associating unit that calculates an occupancy rate of the feature image region relative to an entire image region of the each image to associate a predetermined image display mode with the each image according to the occupancy rate calculated; and
a display control unit that controls to sequentially display the series of images based on the image display mode associated with the each image.

2. The image display apparatus according to claim 1, further comprising an input unit that receives an input of a threshold for the occupancy rate, wherein the associating unit associates a predetermined image display mode with the each image according to a magnitude relation between the occupancy rate and the threshold.

3. The image display apparatus according to claim 1, wherein the image display mode is represented by at least one of a display rate which indicates a speed of display of the each image, and displayability information which indicates whether the each image is to be displayed or not.

4. The image display apparatus according to claim 3, wherein the display rate of the each image successively changes according to the occupancy rate of the feature image region.

5. The image display apparatus according to claim 1, wherein
the predetermined feature includes plural features different from each other,
the region detecting unit detects feature image regions having the respective features from the each image,
the associating unit calculates occupancy rates of the feature image regions having the respective features in the each image, and associates a predetermined image display mode with the each image according to at least one of the occupancy rates calculated.

6. The image display apparatus according to claim 1, wherein
the each image is an image obtained by image capture of an inside of a digestive system, and
the predetermined feature is a feature identifying a mucosa or a foreign substance in the digestive system.

7. The image display apparatus according to claim 6, wherein the foreign substance is at least one of excrement, foam, and mucus.

8. The image display apparatus according to claim 1, wherein
the series of images are generated with a use of a capsule endoscope.
